(19) United States
(12) Patent Application Publication
(10) Pub. No.: US 2025/0230483 A1
(43) Pub. Date: Jul. 17, 2025

(54) OCULAR INSERTS WITH PROTEASE SENSING INDICATORS

(71) Applicant: TearDX LLC, New York, NY (US)

(72) Inventors: Eugene Orloff, New York, NY (US); Paul V. Braun, Champaign, IL (US); Eric Scott Epstein, Pittsburgh, PA (US); Jay Taylor, Urbana, IL (US)

(21) Appl. No.: 19/018,667

(22) Filed: Jan. 13, 2025

Related U.S. Application Data
(60) Provisional application No. 63/620,492, filed on Jan. 12, 2024.

(51) Int. Cl. C12Q 1/37; C12Q 1/04

(52) U.S. Cl. CPC C12Q 1/37; C12Q 1/04

(57) ABSTRACT

An ocular insert containing one or more reporters is described. Each reporter is bound to the ocular insert via a peptide sequence selectively cleaved by a targeted protease. Each peptide sequence is hydrolytically stable in tear fluid for at least 30 minutes in absence of the targeted protease. A biocompatible fluid container containing one or more sensors is described. Each sensor contains reporters bound to one or more surfaces contacting the biocompatible fluid and released from the surface when exposed to specific pathogens. A wearable ocular insert directly capturing biomarkers for ocular disease is described. The biomarkers are detected either once released from the ocular insert or directly onto the surface of the ocular insert. A lateral flow device comprising a modified Schirmer's strip and at least one test region is described. The test region comprises immobilized peptides, antibodies or aptamers, and a lateral flow device weighing no more than 4 g.

### CROSS-REFERENCE TO RELATED APPLICATIONS

This application claims the benefit of U.S. Provisional Patent Application No. 63/620,492, filed Jan. 12, 2024, which is hereby incorporated by reference in its entirety.

### FIELD OF INVENTION

The application is in the field of ocular inserts and medical diagnostic tests.

### BACKGROUND

Diagnostic tests for ocular biomarkers have been reported in academic literature. In a typical test, either a Schirmer's strip, a microcapillary tube, or a swab is used to collect a small amount of tear fluid, and then an assay, such as ELISA or PCR, is conducted to determine either quantitatively or qualitatively the amount of one or more biomarkers of interest in the collected tear fluid. These procedures are time consuming and require specific skills which prevent them from being utilized widely in clinics. Furthermore, the very small volumes of tear fluid that can be practically collected using these methods (on the order of about 1 to 10 microliters), make detection of many biomarkers that are of low concentrations prohibitively challenging to be practical for clinical diagnostics, For example, many biomarkers have a concentration in tears lower than about 1 micromolar and many still are on the order of several nanomolar or less. Therefore, there is a need for improved methods of collecting biomarkers of interest in tear fluid.

Infections of the cornea caused by specific types of viruses, bacteria, and fungi, as well as acanthamoeba, can cause rapid deterioration of the cornea. Failure to accurately diagnose and quickly treat these infections can lead to blindness, loss of the eye, or death. These infections are currently diagnosed by visual inspections, which are prone to human error, or by culturing of corneal scrapings, which is time consuming (taking approximately 72 hours) and has been shown to have a low diagnostic sensitivity. For cases of aggressive, infectious keratitis, there is a need for more accurate and rapid diagnostic tests that are simple and inexpensive enough to be used practically at the point of care.

Many ocular pathogens that cause infectious keratitis, including bacteria, fungi, and acanthamoeba, are known to secrete proteases that are specific to their particular species or strain. Several ocular pathogen-specific proteases have been shown to selectively cleave specific sequences of peptides. This pathogen-specific, selective peptide cleavage can be leveraged to create diagnostic tools with high specificity for a targeted pathogen.

Contact lens wearers, in particular, are prone to infectious keratitis. Users of eye drops are another at-risk population of people for infectious keratitis, as several brands of eye drops have recently been recalled due to contamination with a highly aggressive bacteria, Pseudomonas aeruginosa. Therefore, it may benefit both users and manufacturers of eye drops as well as contact lens solutions, for there to be a simple, convenient, inexpensive means of detecting contaminations by certain known pathogens. It is desirable for such a sensor to be free of any electronics or complex instrumentation.

The mechanism of reporter release by protease-responsive peptides has been demonstrated previously. Such approaches have required either that the reporter be incorporated within a peptide-based fluorescence resonance energy transfer (FRET) fluorophore, which is complex, expensive, and often requires an external device to sense the FRET signal (Chem. Soc. Rev., 2022, 51, 208), or a separate mechanism for separating the released reporter from the sensing region. For example, Safina et al. utilized a magnetic reporter and an external magnet to generate a color change utilizing a protease-responsive peptide bound to the magnetic reporter (Analyst, 2013, 138, 3735). Such devices are practical only as non-wearable diagnostic sensors. In cases of diseases of the eye, such as with viral, bacterial, or fungal infections, the concentration of proteases released by these pathogens in the tear fluid may be too low to detect using non-wearable diagnostic sensors.

Therefore, a diagnostic sensor is disclosed, comprising a wearable ocular insert, on which a sensor is adhered containing a peptide-bound reporter, where the reporter is a biocompatible dye, pigment, particle, or bead, and the peptide is covalently bound to the surface of the ocular insert, either directly or indirectly through a polymer that is bound to the surface of the ocular insert.

Binding the peptide and reporter to an ocular insert enables more sensitive detection of proteases because the ocular insert can be worn for extended periods of time and, therefore, can enable larger quantities of protease to access the peptides than may be practical by utilizing a conventional eye swab. In addition, the use of a wearable ocular insert can enable effective dissipation of the reporter without the use of an external or complex mechanism, as has been previously required, because the reporter can be released directly into the tear fluid within the eye, which is continuously turned over through natural blinking.

Biomarkers in the tear film and the ocular surface are an underutilized source of information for diagnosing ocular diseases. One of the biggest challenges that prevents the biochemistry of the ocular surface from being tested in clinicals settings is the difficulty of obtaining and handling tear samples or corneal scrapings from patients. Typically, tear fluid is collected either using strips of paper known as Schirmer's strips, cotton swabs, or microcapillary tubes. All of these methods collect only small volumes of tears (typically several microliters) and also risk generating reflex tears during collection which can have drastically different composition than basal tears. Consequently, the concentration of biomarkers of interest in tear fluid are often too low to detect or highly variable. Furthermore, the conventional tear sampling methods also require advanced protocols to separate the biomarkers of interest from the rest of the proteins, lipids, cells and other components that are collected. Thus, from a practical perspective, these require a special lab to conduct tests. It may be of great benefit to both patients and clinicians to develop ocular diagnostic tests that can be conducted easily and inexpensively at the point of care. However, in order to make an ocular biomarker test practical for use at the point-of-care, there is a need for a simplified and effective means of sampling the biomarkers of interest in tear fluid.

Each year, ocular Herpes Simplex Virus (HSV) keratitis infects roughly 1.5 million new patients globally and accounts for 40,000 new cases of severe visual impairment in the U.S. Early diagnosis and treatment of ocular HSV infections (within the first 72 hours) helps prevent irreversible damage to the eye and can help prevent reactivation events, which over time cause vision impairment through corneal scarring. It is reported that 27% of all people with an initial infection will have another outbreak within 1 year and 50% will have another outbreak within 5 years of initial infection. Each reactivation event significantly increases the risk of damage to the eye. The ability to quickly and accurately diagnose HSV infection and reactivation remains a significant challenge for optometrists and is crucial to preventing visual impairment and blindness.

Serological assays for HSV, which do exist, have only limited utility for HSV keratitis detection because the majority of people will test positive for HSV-1 immunoglobulins regardless of whether or not an infection is active. Therefore, there is a need for tests that detect an actual outbreak of the HSV-1 virus. It is known that HSV-1 virions shed in the tear fluid of mice during an active ocular HSV-1 outbreak. This disclosure presents embodiments of diagnostic tests suitable for detecting many ocular biomarkers, including, but not limited to, proteins on the HSV-1 viral envelope.

Lateral flow assays (LFA) are a simple, convenient tool for diagnosing a variety of infections. Despite their ease of use, there are very few commercially available LFAs for diagnosing ocular infections. Among the main challenges with applying LFAs to a variety of ocular conditions are the challenges associated with sampling tear fluid. Oftentimes the amount of tear fluid collected is too little and the concentration of the target biomarker is beneath the detection limit of the LFA making analysis difficult. Other times, due to inconsistencies with clinicians collecting the sample, the quality of the tear sample is too poor to accurately assess the composition of the tear film. In other words, the signal-to-noise ratio required for naked eye detection in a lateral flow assay may not be achievable for many biomarkers of interest in tear fluid using existing tear sampling methods.

### SUMMARY OF THE INVENTION

The present invention includes an ocular insert containing one or more reporters, wherein each of the one or more reporters is bound either directly or indirectly to the ocular insert via a peptide sequence that is selectively cleaved by a targeted protease, wherein each protease-cleavable peptide sequence is hydrolytically stable in tear fluid for at least about 30 minutes in the absence of the targeted protease.

The present invention includes a biocompatible fluid container with one or more passive sensors that present an optical or photoacoustic signal that can be detected either by the naked eye or by an ultrasound probe when in contact with a known pathogen. Importantly, the passive sensor is small enough to be contained within the biocompatible fluid container. Such biocompatible fluid containers include, but are not limited to, contact lens cases and eye drop solution bottles. Each sensor contains reporters bound to one or more surfaces that contact the biocompatible fluid, wherein the reporters are released from the surface when exposed to a specific pathogen. Each sensor may be in the form of a single, or multiple, beads, films, gels, membranes or strips. In some embodiments, the mechanism of reporter release is via cleavage of a specific peptide sequence by a protease that is secreted by the targeted pathogen.

The present invention includes devices and methods for improving the sampling of targeted biomarkers in tear fluid as a means to improve the signal-to-noise ratio of point-of-care diagnostic tests for ocular diagnostics including lateral flow assays. The invention is comprised of a wearable ocular insert, which contains a covalently-bound capture agent that selectively targets the biomarker of interest in tear fluid. The ocular insert is potentially combined with a release agent, which releases a component of the biomarker of interest (referred to here as the analyte). The released analyte is then detected by a point-of-care assay. The analyte may be a component of the captured biomarker or may be the entire captured biomarker, which has been released from the ocular insert.

In preferred embodiments, the wearable ocular insert is either a contact lens, punctal plug, or of a form similar to a Schirmer strip; however, the wearable ocular insert may be any object that can readily be placed and worn for extended periods of time (at least about 5 minutes) either on the surface of the eyeball or inside the conjunctival sac or lacrimal punctum of the human eye.

In preferred embodiments, the targeted biomarker is captured and localized to a particular region within the ocular insert, and when the insert is exposed to an indicator solution, the captured biomarker becomes detectable. The indicator solution contains biological molecules (for example, antibodies, aptamers, or proteins) conjugated to a visualization agent (for example, gold nanoparticle, dyed polystyrene particle, fluorescent label, quantum dot, or photoacoustic label) that binds specifically to the captured biomarker and provides a detectable signal indicating the presence of the biomarker.

In other preferred embodiments, the assay is a lateral flow assay comprised of capture and detection agents that detect at least one component of the released component of the biomarker captured by the ocular insert. The use of a wearable ocular insert containing immobilized binding agents enables the targeted biomarkers of interest to be collected and concentrated for as long as the ocular insert is worn by the patient. This prolonged collection and concentration of biomarkers greatly enhances the signal-to-noise ratio of the subsequent assay.

This invention is particularly well suited for improving the detection of ocular antigens such as viruses, bacteria, fungi, or amoeba. For example, viruses, such as the HSV-1 virus, contain many glycoproteins that are bound to the viral envelope. These glycoproteins (the analyte of interest) may be directly targeted by antibodies contained within an LFA; however, reliable detection of the virus in this way is challenging because many of the targeted glycoproteins may not be accessible to the antibodies of an LFA while the virus remains intact. Many whole pathogens that infect the ocular surface are relatively large in size (>200 nm) compared to most antigens targeted by LFAs (<50 nm). For this reason, the pathogen may either be broken up prior to introduction through the LFA or directly detected on the ocular insert. Therefore, it is preferable to first release the targeted analyte from the virion so it may more easily be captured and detected by the antibodies on an LFA. As a non-limiting example, FIG. 20 describes the release of the analyte by lysing the captured virus in a lysis buffer. This example illustrates the invention as the combination of an antibody-coated ocular insert, a lysis buffer, and an LFA device. As another example, the direct detection of the pathogen on the ocular insert, through the use of an antibody conjugated nanoparticle may be described herein.

### BRIEF DESCRIPTION OF THE DRAWINGS

FIG. 1 is an example of a single sensor region attached to the surface of a contact lens;

FIG. 2 is an example of multiple sensor regions attached to the surface of a single contact lens, each of which targets a different ocular pathogen;

FIG. 3 is an example of representation of a sensor region attached on the surface of a contact lens in an annular shape around the lens itself;

FIG. 4 is an example of a sensor region occupying a larger section of a contact lens;

FIG. 5 is an example of a contact lens architecture containing a blue-colored hydrogel that degrades in the presence of a protease secreted by a bacteria, revealing a different red-colored architecture underneath;

FIG. 6 is an example of a contact lens with the entire backside containing a protease-responsive peptide/reporter system;

FIG. 7 is an example of a contact lens containing a protrusion to enable insertion and removal of then contact lens without touching the surface;

FIG. 8 is an example of a punctal plug with a protease-reporter system bound to the surface of the punctal-plug bulb;

FIG. 9 is an example of a punctal plug with a protease-reporter system bound to the surfaces of both the bulb and inner channel of the punctal-plug;

FIG. 10 is an example of a punctal plug with a protease-reporter system adhered to a permeable material that is inserted into the inner channel of the punctal plug;

FIG. 11 is a first example of a modified Schirmer's strip with a protease-response reporter that will change color when exposed to a targeted protease;

FIG. 12 is another example of a modified Schirmer's strip with a protease-responsive reporter that will change color when exposed to a targeted protease;

FIG. 13 is an example of when an ocular insert has a surface containing two reporters (A and B), whereby when the surface comes into contact with a targeted protease, only one reporter (B) is cleaved and removed from the surface, thereby exhibiting a detectable change to the surface.

FIG. 14 is an example of when a contact lens has a permanently pigmented or dyed material embedded in a surface and a second layer of material containing a reporter is bonded to the surface, such that when the second layer of material is exposed to the targeted protease and is cleaved and removed, a detectable change to the surface is exhibited;

FIG. 15 shows the results of a set of experiments illustrating the release of a fluorescent dye molecule in the presence of a protease where the fluorescence intensity of the surrounding solution increases by both increasing protease concentration and time exposed to the protease;

FIG. 16 is an example of cleavable reporters covalently bound to a water-permeable material via cleavable peptide bonds, whereby the permeable material is placed on top of the surface of an ocular insert;

FIG. 17 is an example of a porous, branched polymeric structure containing reporters covalently bound to the branched polymer via cleavable peptides;

FIG. 18 is an example of proposed chemistry linking a colored pigment to the surface of a silicone ocular insert;

FIG. 19 is an example where a biocompatible fluid container contains reporters bound to beads via a peptide sequence that is cleaved selectively in the presence of a protease, where upon peptide cleavage, the reporters are released into the solution, causing the color of the solution to change in a way that is either readily detected by the naked eye or with the aid of an additional instrument, such as a UV or visible flashlight in cases where the reporter is a fluorescent or luminescent moiety, such as -Ahx, Gly-Gly-Gly-Ahx-Cys-, which is selectively cleaved by the LasA protease;

FIG. 20 is an example of a color change through protease cleavage of a chromogenic substrate;

FIG. 21 is an example of a capture-and-release strategy for capturing HSV-1 virions and releasing HSV-1 analyte for detection on an LFA;

FIG. 22 is an example of capturing and detecting HSV-1 virions directly onto the ocular sampling tool through an antibody conjugated nanoparticle:

FIG. 23 shows the results from a set of experiments showing how the general strategy outlined in FIG. 22 may be used to bind and detect HSV-1 virions on the surface of a prototype ocular insert after performing a simple immunoassay on the ocular insert after it was exposed to a series of solutions containing HSV-1 virions;

FIG. 24 is an example of a porous membrane containing covalently-bound capture antibodies laminated to the surface of an ocular insert;

FIG. 25 is an example of capture antibodies immobilized on a roughened surface on a portion of an ocular insert;

FIG. 26 is an example of a colorimetric or fluorometric test region attached to the surface of a contact lens that will undergo a change in appearance if the region has captured the targeted biomarker;

FIG. 27 is an example of multiple colorimetric indicators attached to the surface of a single contact lens, each of which targets a different ocular pathogen and is revealed by a colorimetric or fluorescent assay;

FIG. 28 is an example of a colorimetric indicator on the surface of a contact lens in an annular shape around the lens itself that will change appearance if exposed to a targeted biomarker;

FIG. 29 is an example of a test region occupying a larger section of a contact lens that will change appearance if exposed to a targeted biomarker;

FIG. 30 is an example of a contact lens with a concave side containing the embedded test and control regions that will change appearance if exposed to a targeted biomarker;

FIG. 31 is an example of a contact lens containing a protrusion to enable insertion and removal of contact lens without touching the surface and that will change appearance in the presence of a targeted biomarker; and

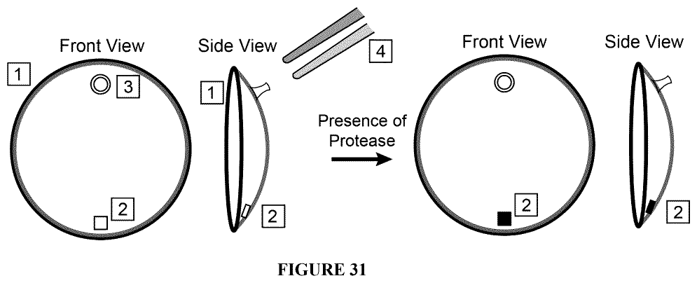

FIG. 32 is an example showing an LFA Schirmer's strip.

### DETAILED DESCRIPTION

Various ocular inserts and containers including one or more reporters are described herein. Each reporter may be bound to the ocular insert or container via a peptide sequence selectively cleaved by a targeted protease.

For purposes of this disclosure, a contact lens may refer to any polymeric object that can fit conformally to the surface of the eyeball. The contact lens in this context need not be optically transparent nor act as an optical lens, as may be required for more traditional uses of a contact lens. The thickness of the contact lens may vary between about 50 microns to about 2 mm. The contact lens may contain one or more sensors to detect one or more different proteases. In cases where more than one sensor is embedded in the contact lens, they may preferably be separated by a distance large enough to be readily distinguishable by the naked eye. The shape of each sensor may be, for example, rectangular, circular, or an annual ring around the contact lens or a portion thereof, or any shape that is large enough to be observed by the naked eye (at least about 50 microns in observable area).

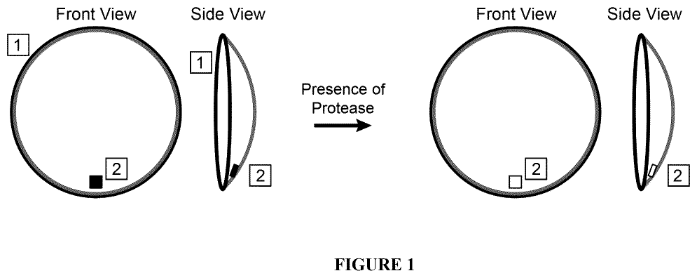

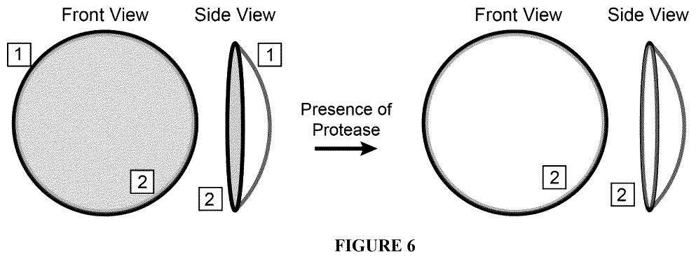

Several examples of different layouts of the protease-responsive sensors are shown in FIGS. 1-7. In some embodiments, one or more sensors are embedded on the convex surface of the contact lens. In other cases, it is preferable for the sensor to be on the concave side of the lens that directly contacts the eye (as shown in FIG. 6). Such an architecture is particularly preferable for cases where the contact lens is to be used as a point-of-care device for diagnosing corneal ulcers. Embedding the sensor on the concave surface that directly contacts the epithelium of the eyeball enables the sensor to be placed in direct contact with, or in the immediate vicinity of, the inflamed surface of the eye. This architecture greatly simplifies the procedure required to sample corneal ulcers, which traditionally are sampled for lab culture by first scraping the top surface of the cornea or conjunctiva where the ulcer is observed. Placing the one or more sensors on the concave side of the contact lens circumvents the need for corneal scraping as it comes in direct contact with the eye. For purposes of simplifying the manufacturing, it may be preferable for one peptide-based sensor to coat the entire (both concave and convex) surface of the contact lens.

Contact-lens wearers, in particular, are prone to infectious keratitis. Therefore, in certain cases, it is desirable for a traditional contact lens (i.e., one that is transparent and may or may not focus light) to have an inexpensive sensor for known pathogens. For this specific application and in order to avoid impacting the wearer's vision, the sensor may be added to a surface sufficiently far away from the area of the contact lens where light enters and focuses towards the retina.

FIG. 1 is an example of a single sensor region (2) unique to a single protease attached to the surface of a contact lens (1). Although not shown, in preferred embodiments, the contact lens contains an additional control sensor region, preferably with reporters bound by peptides that are not cleavable by the targeted protease. When the contact lens is exposed to the selected protease, the appearance of the sensor region (2) will change, becoming transparent in this case.

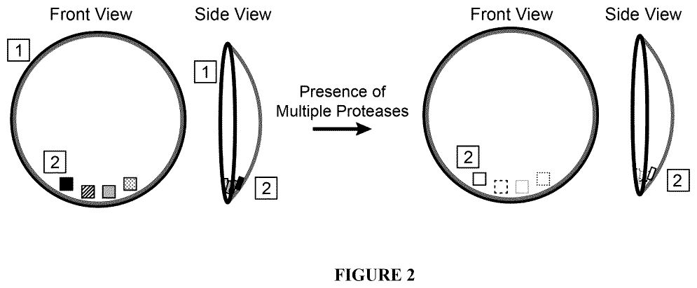

FIG. 2 is an example of multiple sensor regions (2), each responsive to a unique protease and each of which targets a different ocular pathogen attached to the surface of a single contact lens (1). When each sensor interacts with its specific protease, the sensor will change its appearance.

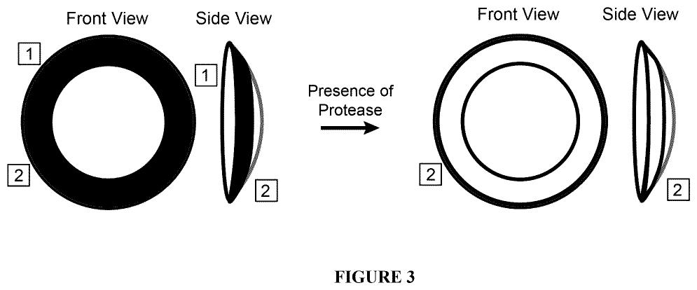

FIG. 3 is an example where the sensor is designed to be an annular sensor region (2) around the surface of a contact lens (1).

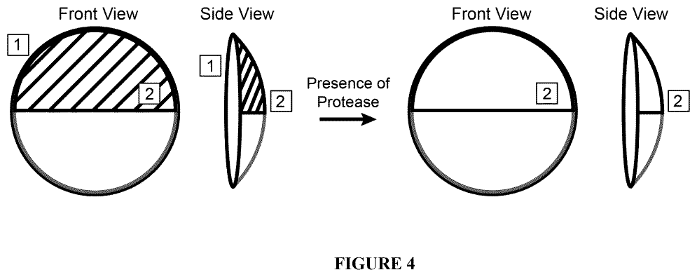

FIG. 4 is an example of the peptide/reporter sensor region (2) occupying a larger section of the contact lens (1), which may be anywhere from 10%-100% of the total area of the lens.

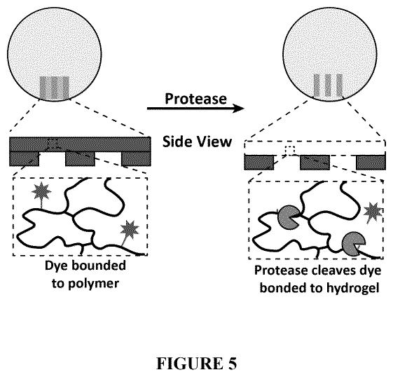

FIG. 5 is an example of a contact-lens architecture containing a blue-colored hydrogel that degrades in the presence of a protease secreted by a bacteria, revealing a different red-colored architecture underneath.

FIG. 6 is an example of a contact lens (1) with an unmodified lens surface (front, convex, side) and with the entire backside (2) containing a protease-responsive peptide/reported system on the concave surface, with covalently-bound peptides and reporters. In the presence of an infection agent, the modified surface may undergo a response, changing its initial appearance.

In some embodiments, the ocular insert may contain a permanently colored layer of material. This layer may consist of a dyed or pigmented polymer, a layer of inorganic material (such as a colored ceramic or metal), or dye or pigment particles that are bound to a surface of the ocular insert. In some embodiments, this permanently colored layer may be a portion of the volume of the ocular insert itself which has been injected with or polymerized around a dye or pigment. In some embodiments, this layer may be designed to take on a specific shape to make it more easily recognized.

For example, in FIG. 5, the permanently-colored material is displayed as three red-colored bars. A second layer is then positioned on top of the first layer, which displays a visible color different from the first layer. This material is comprised of a specific peptide sequence that cleaves in the presence of a pathogen-specific protease. Upon cleavage, this color of the second material diffuses out from the contact lens, thereby revealing the permanently colored layer beneath it. Preferably, the change in color is detectable by the naked eye so as to alert the user to an infection or contamination of the medium within which the ocular insert is stored (typically, either the eye or in a contact-lens storage solution).

In the example in FIG. 5, this second degradable layer is shown as a blue colored hydrogel that is crosslinked by the protease cleavable peptide. Upon cleavage, hydrogel dissolves, thereby revealing the red colored bars underneath. In some embodiments, it is preferable for the second colored layer to have a different shape than the first layer, thus making the change readily recognizable, for example, to those that may be color blind.

In some embodiments, the ocular insert may contain embedded magnetic nanoparticles to aid the user with inserting and removing the ocular insert from the eye using a magnet.

In some embodiments, the contact lens may be designed to contain a protrusion extending outward from the convex surface of the contact lens to enable easy insertion in the eye without directly touching the surface of the contact lens which contains the protease-specific peptides and reporters. To further aid with the insertion and removal of the lens without damaging or contaminating the sensor region, the ocular insert may contain permanently embedded magnetic particles, such as iron oxide. Such a contact lens may be inserted and removed using any object with a magnetic tip.

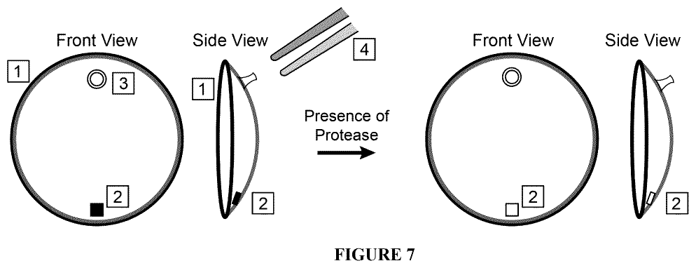

FIG. 7 is an example of a contact lens (1) containing a protrusion (3) to enable insertion and removal of contact lens without touching the surface, for example by the ends of tweezers (4). The contact lens is provided with a sensor (2), which may be a colorimetric indicator, in which its appearance is altered when in the presence of the targeted protease.

In cases where tear fluid needs to be sampled for extended periods of time, it may be advantageous to place the sensor on a punctal plug. A punctal plug refers to any object designed to be inserted into the lacrimal punctum. Punctal plugs are typically comprised of a polymer, such as silicones, collagen, or a hydrogel. Punctal plugs are typically approximately cylindrical objects with a diameter between about 100 microns to about 1 mm, and a length between about 1 mm and about 5 mm.

In some embodiments, the punctal plug may be dyed or pigmented to display one color, and may incorporate a peptide bound dye or pigment of another color. In some embodiments, the punctal plug contains one or more channels extending through the length of the punctal plug, thereby enabling tear fluid to continuously flow through the plug into the lacrimal punctum. In some embodiments, the punctal plug may contain one or more channels open at the top of the plug and closed at the bottom of the plug so as to increase the available volume in which to place a protease-responsive sensor, but such that tear fluid does not flow through the plug into the lacrimal punctum. The use of an open or closed channel is particularly useful for protease sensors with either light-absorbing or fluorescent readouts, as the channel serves to increase the optical depth and thereby the color contrast, making it easier to detect a color change by eye.

For example, the channel may be filled with pigments, beads, or particles of one color, while the top bulb of the plug contains the protease-responsive, peptide-bound reporter of another color. As another example, the punctal plug may be dyed or pigmented one color, and a channel running through the punctal plug may be filled with another material (e.g., a membrane or beads) containing the protease-responsive, peptide-bound reporter of another color. This embedded material need not be chemically bonded to the inner surface of the punctal plug as long as it is mechanically stable within the open channel. For example, a porous thread or strip of cellulose may be embedded inside of the channel of the punctal plug, while the protease-responsive, peptide-bound reporters are chemically bound to the cellulose.

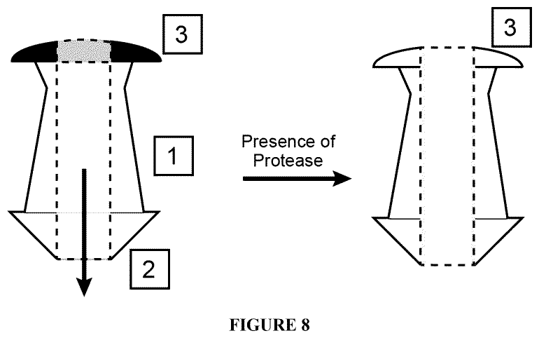

FIG. 8 illustrates a punctal-plug insert (2), with a channel running through the insert (1) allowing tear fluid to flow through the insert and exit, and a protease-responsive peptide and reporter is bound to the top bulb of the punctal-plug surface (3). The reporter is released from the bulb in the presence of an infectious agent that secrets the targeted protease. The released reporter is carried away by the continuous turnover of tear fluid in the eye changing the appearance of the top bulb of the punctal-plug surface (3).

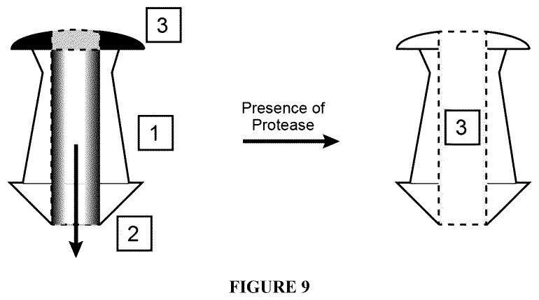

FIG. 9 illustrates a punctal-plug insert (2), with a channel running through the insert (1) that allows tear fluid to exit, and protease-responsive peptide and reporter that is bound to both the top bulb (3) and inner channel (1) of the punctal plug. The reporter is released from the bulb and channel surfaces in the presence of an infectious agent that secrets the targeted protease. The released reporter is carried away by the continuous turnover of tear fluid in the eye changing the appearance of the bulb and inner channel (3).

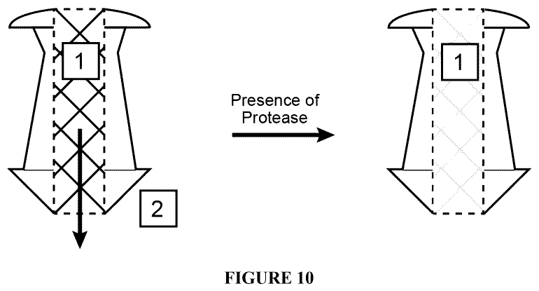

FIG. 10 is an example of a punctal plug (2) with permeable material (1) containing a peptide-bound reporter (represented by the black grid) and a channel running through the insert that allows tear fluid to exit the peptide-reporter system adhered to the permeable material that is inserted into the inner channel of the punctal plug. Examples of useful permeable materials for this application include, but are not limited to, a network of peptide-bound beads and a cellulosic membrane. In the presence of an infectious agent, reporter is released from the material and flows out of the punctal plug changing the appearance of the permeable material (1).

In some embodiments, the ocular insert may be a Schirmer's strip, which is a water-permeable strip of material designed such that one end may be inserted into the conjunctival sac. While inserted, tear fluid may wick into the material and flow along the length of the strip via capillary action. The Schirmer's strip may be comprised of any water-permeable polymeric material, but preferably is comprised of cellulose or derivatives of cellulose (e.g., oxidized cellulose, cellulose esters, or nitrocellulose). In this modified version of a Schirmer's strip, a section of the Schirmer's strip may be chemically modified to contain a test region comprised of one or more protease-responsive reporters. Preferably, this test region is positioned within a distance of 0 mm to about 20 mm away from the end of the strip to be inserted into the conjunctival sac. A Schirmer's strip is advantageous because it is widely utilized by, and therefore familiar to, optometrists and ophthalmologists. However, the peptide-bound ocular insert can be any object that is readily inserted or removed from the conjunctival sac. For example, instead of a Schirmer's strip, the insert can be comprised of a string of cellulosic material that is modified with the peptide-bound reporter. Alternatively, it may be a disc or crescent shaped hydrogel that is crosslinked solely by the protease-responsive peptides (similar to the material illustrated in FIG. 5).

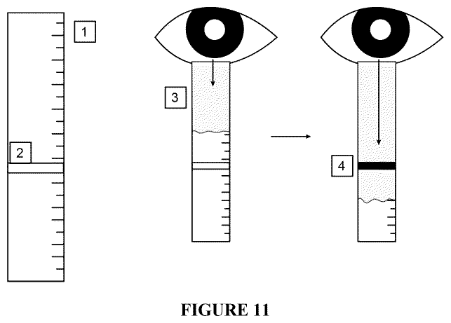

FIG. 11 is a first example of a modified Schirmer's strip (1) with a protease-response reporter (2) that will change when exposed to the targeted protease. In this example, the peptide-bound reporters are immobilized in a test region positioned at a distance away from the conjunctival sac (for example, at least 5 mm). Here, capillary action enables tear fluid to flow down the strip (3) and remove any reporters that have been released via protease-induced cleavage of the immobilized peptides. When the strip is inserted into the conjunctival sac and tears flow down the Schirmer's trip, interaction between tear fluid and a chemically modified test line (2) comprised of protease-responsive reporters will affect the test line such that the presence of a protease can be determined (shown as 4).

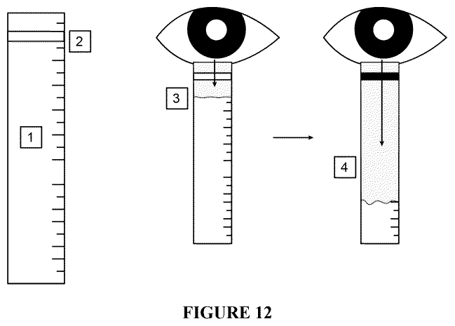

FIG. 12 is another example of a modified Schirmer's strip (1) with a chemically-modified test region comprised of protease-responsive reporters (2) that will change when exposed to the targeted protease (shown as 4). In this example, the peptide-bound reporters are immobilized in a test region positioned such that it is fully embedded within, or immediately adjacent to, the conjunctival sac (3). This architecture is advantageous when the peptides need maximum exposure to the protease in tear fluid. Both the natural turnover of tear fluid in the eye as well as capillary action down the strip are able to remove any reporters released as a result of protease-induced cleavage of the immobilized peptides. The region is positioned to be embedded within the conjunctival sac of the eye. Capillary action may cause tear fluid to flow down the length of the strip, but here is not required for the peptide-bound reporters to be released, as the test region is placed within the conjunctival sac.

In some embodiments, the protease-responsive reporter may be selected so as to be readily detectable by the naked eye. Such detection agents include dyes, pigments, particles, or beads that are light-absorbing, fluorescent, chemiluminescent, or have a high reflectance in a portion of the visible-to-near IR spectrum. In other embodiments, the reporter may be selected so as to be detected by a photoacoustic imaging device.

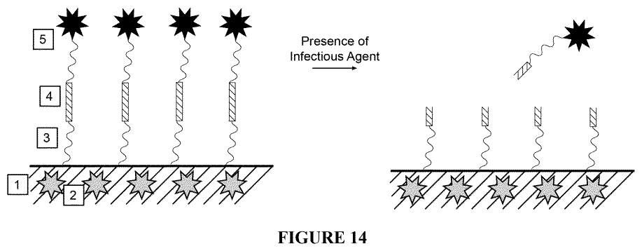

The reporter may be covalently bonded directly to one end of the protease-responsive peptide, or to a polymer which is bound to the protease responsive peptide and the surface of the ocular insert. In these embodiments, when the peptide is cleaved by the protease, the reporter is free to diffuse out of the sensor region, thereby revealing the color and/or photoacoustic signal of the ocular insert in the absence of the reporter (as illustrated by FIGS. 14-18). In other embodiments, the reporter may be bound to a hydrogel which is crosslinked by the protease-responsive peptide. When the peptide is cleaved by the protease, the crosslinked gel dissolves, thereby revealing the color or photoacoustic signal of the ocular insert in the absence of the hydrogel-bound reporter (as illustrated in FIG. 5). It may be advantageous to construct the protease-responsive layer out of a hydrogel as the thickness and, therefore, the volume (and in cases of optical reporters, the optical depth) can be tailored to increase the signal of the reporter. Furthermore, the mesh size and thickness of the hydrogel can be tailored to optimize the rate at which the color change occurs.

In some embodiments, it may be desirable to covalently bind the reporters as a monolayer (or several monolayers) directly on the exposed surface of the ocular insert, as this will maximize the accessibility and, thus, the response of the peptide-bound reporters to the targeted protease. The top-most surface may be the surface of the ocular insert, an additional polymer, or an inorganic material that is strongly adhered to an exposed surface of the ocular insert.

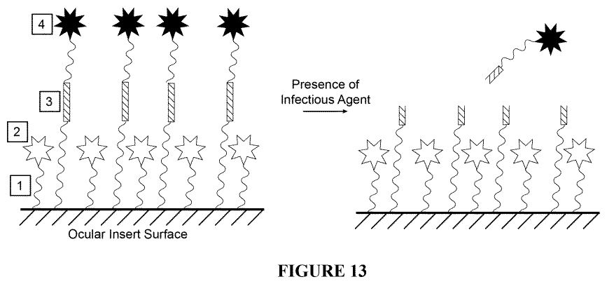

FIG. 13 is an example of when the surface of the ocular insert (1) contains two reporters (A and B, shown as 2 and 4, respectively), whereby when the surface comes into contact with a targeted protease, only reporter B is cleaved and removed from the surface, thereby exhibiting a detectable change to the surface. For example, reporter A may be covalently bound to the surface of the ocular insert via any covalent linkage that is not susceptible to cleavage under conditions during which the ocular insert may be in contact with tear fluid or another biofluid (1), and Reporter B may be covalently linked to the surface of the ocular insert via a peptide sequence sensitive to an infection agent (3). Optionally, the covalent linkages may contain a biofouling resistant polymer, such as polyethylene glycol.

FIG. 14 includes an ocular insert surface (1), reporter A (2) embedded within the polymeric matrix of the ocular insert, a covalent bond to the surface of the ocular insert (3) that links a peptide sequence cleavable by the targeted protease (4), and reporter B (5), which is covalently bound to the cleavable peptide sequence. In this example, the surface of the contact lens is a permanently pigmented or dyed material and bonded to the surface of that material is a second layer of material containing a reporter, which when exposed to the targeted protease is cleaved and removed, thereby exhibiting a detectable change to the surface.

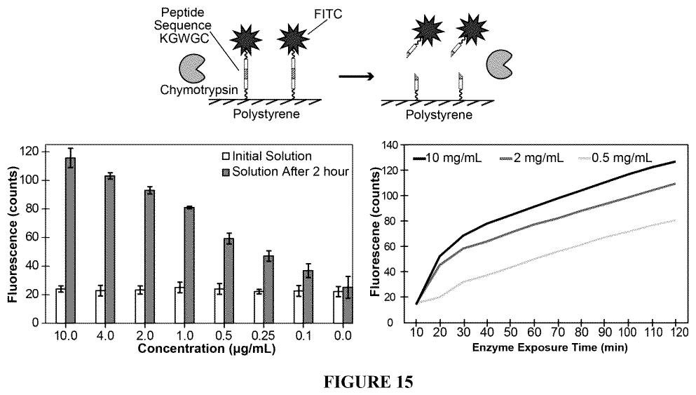

As an example of this type of chemistry and reporting mechanism, the surface may be a modified polystyrene microtiter plate with branched polyethyleneimine, which may yield a surface coated with available primary amines. To this surface, EDC/NHS may be used coupling chemistry to covalently link the C-terminus end of a short peptide with the sequence KGWGC (written N to C terminus). Afterwards, the peptide modified surface may be reacted with a buffered solution containing fluorescein isothiocyanate (FITC) dye that covalently reacts with either the N-terminus end of the peptide or the Lysine (K) residue on the N-terminus side of the molecule. After washing excess dye and peptide from the wells, a surface may be obtained where a fluorescent dye is tethered to the surface of the microwell via a peptide sequence that is readily cleaved by the enzyme chymotrypsin. The chymotrypsin cleaves the peptide on the C-terminus side of the tryptophan (W) group on the peptide. The described surface may exhibit little fluorescence prior to exposure to the enzyme. When FITC dye molecules are in close proximity to one another they are known to self-quench, which minimizes observed fluorescence. Upon exposure to the enzyme, the dye is released from the surface and enters the solution resulting in larger fluorescence intensity. This trend is observed in FIG. 15. The fluorescence intensity is observed to increase with exposure to a higher concentration of and longer exposure to the enzyme.

In cases where the signal of the reporter needs to be increased beyond what a monolayer or multilayer of peptide-bound reporters can achieve on one surface of the ocular insert and, at the same time, the rate of cleavage of the peptides needs to be maximized similarly to what can be achieved with a monolayer or multilayer of peptides, it may be preferable to infuse and bond the peptide-bound reporters within a highly-permeable membrane. In these embodiments, the thickness of the membrane may be tuned to achieve the high signal needed for easy detection (e.g., by the naked eye), while the high permeability of the membrane enables easy access and, thus, rapid response to the pathogen-specific protease. It is preferable in this case for the pore size of the membrane to be much greater (for example, at least about 10 times greater) than the hydrodynamic radius of the protease, as well as much greater than the size of the reporter molecules or particles, so as to enable easy mobility of the reporters out of the membrane after the peptide is cleaved by the targeted protease.

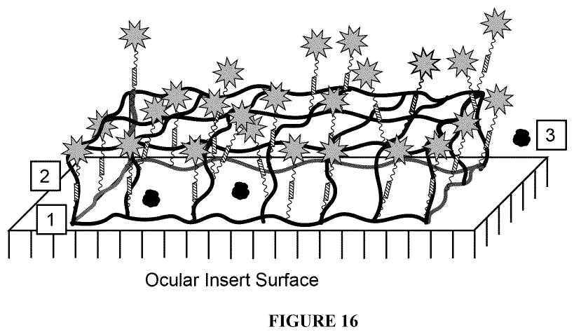

FIG. 16 illustrates a permeable member (2) bonded to the surface of the ocular insert (1) and a reporter covalently bonded to a cleavable peptide which is covalently bonded to surfaces throughout the permeable member, where a protease (3) secreted by the infecting agent is easily able to diffuse through the membrane because the pore size of the membrane is much greater than the size of the protease. In this example, the cleavable reporters are covalently bound to a water-permeable material via cleavable peptide bonds, whereby the permeable material is placed on top of the surface of an ocular insert. The thickness of the permeable material can be tuned to increase the density of the reporter and thereby increase the signal when the reporter is cleaved. In preferred embodiments, the pore size of the permeable material is significantly greater than the size of the targeted protease, such that the protease can access all internal surfaces of the material. In some embodiments, the entire ocular insert may be comprised of this permeable material rather than having the permeable material as a component of the ocular insert, as is illustrated in FIG. 16.

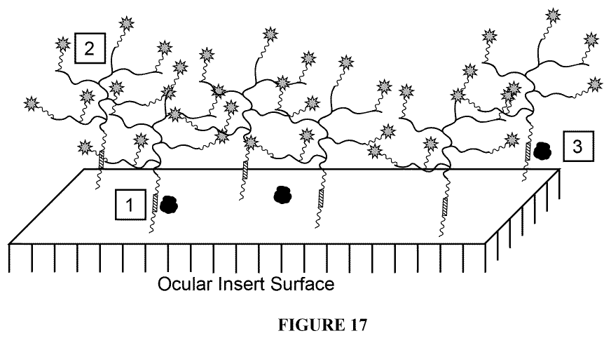

FIG. 17 illustrates a cleavable peptide unit (1), a branched molecular structure (2) containing multiple reporters, and a protease (3) secreted by the targeted infecting agent capable of diffusing through the membrane. In this example, the porous, branched polymeric structure containing reporters is covalently bound to the branched polymer via cleavable peptides.

In one embodiment of this system, the peptide is designed to be cleaved by the LasA protease produced by the bacteria Pseudomonas aeruginosa. This particular protease cleaves amino acid sequences containing-glycine-glycine-glycine-amino acid residues in their structure.

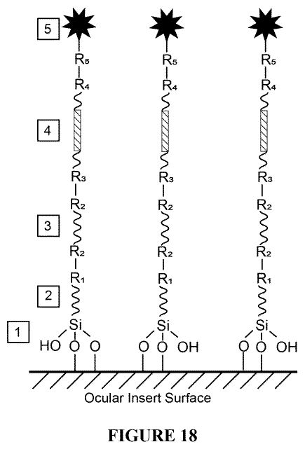

There are several coupling chemistries capable of immobilizing the different components (e.g., ocular insert, peptide, reporter) of the protease-responsive sensor together. As a non-limiting example, a silane coupling agent can be reacted to the surface of a silicone-based polymer, where the silane coupling agent contains an amine end group (FIG. 18, R1) and reacts to a PEG polymer with an NHS ester end group (FIG. 18, R2). On the other end of the PEG polymer is a maleimide functional group (FIG. 18, R3) that reacts to a peptide sequence ending in a cysteine residue containing an available thiol group (FIG. 18, R4). The other end of the peptide contains a primary amine group (FIG. 18, R5) that can be coupled to a dyed species with available carboxylic acid groups via EDC/NHS chemistry or similar carbodiimide coupling chemistries. The product of each of these chemical reactions are hydrolytically stable groups (e.g., amide or thiosuccinimide) that will anchor the reporter to the contact lens under mild conditions. The use of silane coupling agents is particularly useful for bonding the sensor elements to silicone or any material containing hydroxyl groups. In some embodiments, it may be preferable to first chemically modify the surface of the sensing surface before bonding the peptide, reporter, and other useful moieties to that surface. As a non-limiting example, a cellulosic Schirmer's strip or membrane may first be oxidized using sodium periodate to form aldehyde groups.

FIG. 18 is a non-limiting example of a chemistry linking a reporter to the surface of a silicone ocular insert. The surface of the silicone insert is coupled to a polymeric spacer using an amine functional silane chemical reagent coupling agent (1) which is covalently bound to a polymeric spacer (3) via an amide bond (R1-R2). The other end of the polymeric spacer may contain a maleimide functional group (R3) which may be covalently bonded to a protease-responsive amino acid sequence (4) that contains a cysteine residue. The other end of the protease-cleavable amino acid sequence contains an amine group (R5), which may be bound to a carboxylic acid functionalized reporter (5).

The specific combined architectures and chemistries disclosed herein may be used in a preferred embodiment as a point-of-care diagnostic device. These ocular sensors are particularly suited for diagnosis of Pseudomonas aeruginosa or Staphylococcus aureus, which are the two most common bacteria causing microbial keratitis.

Sensors for detecting Pseudomonas aeruginosa may contain the peptide sequence,-Gly-Gly-Gly-, which may be cleaved selectively by the LasA protease, which is secreted by Pseudomonas aeruginosa. In preferred embodiments, the Gly-Gly-Gly sequence is elongated so as to enable easy integration into the sensor. An example of a suitable elongated peptide sequence is NH2-Ahx-Gly-Gly-Gly-Ahx-Cys. Alternatively, a different peptide sequence may be chosen to be cleaved by any of the other proteases that are secreted by Pseudomonas aeruginosa. In addition to LasA, these include Pseudomonas aeruginosa elastase, LasB, protease IV, phospholipase C, and exotoxin A.

For detection of proteases secreted by Staphylococcus aureus, one may use the peptide sequence ETKVEENEAIQK. In preferred embodiments, this sequence may be elongated to enable easy integration into the sensor. An example of a suitable elongated peptide sequence is NH2-Ahx-ETKVEENEAIQK-Ahx-Cys.

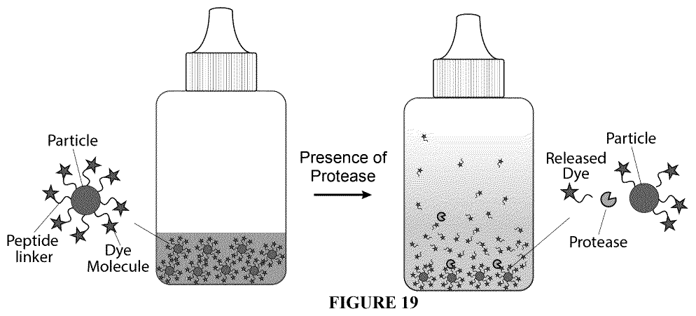

FIG. 19 is an example of a biocompatible fluid container that contains reporters bound to beads via a peptide sequence that is selectively cleaved by a protease. Such a container can be used to give a visual indication when the fluid is contaminated. In some embodiments, the reporters may be a dye that is soluble in the biocompatible fluid such that, upon release, the solution substantially changes color in a way that can be detected by the naked eye. In preferred embodiments, the material to which the reporters are bound is either permanently bonded to one or more surfaces of the biocompatible fluid container or, for cases of fluid dispensers, are larger than the opening of the biocompatible fluid dispenser. An example of this type of system may be where a polystyrene microparticle is conjugated with the following peptide sequence-Ahx,Gly-Gly-Gly-Ahx-Cys-, linking it together with a dye. This particular sequence, which contains three consecutive glycine residues, is known to be cleaved by the LasA protease, a key biomarker found in Pseudomonas aeruginosa infections.

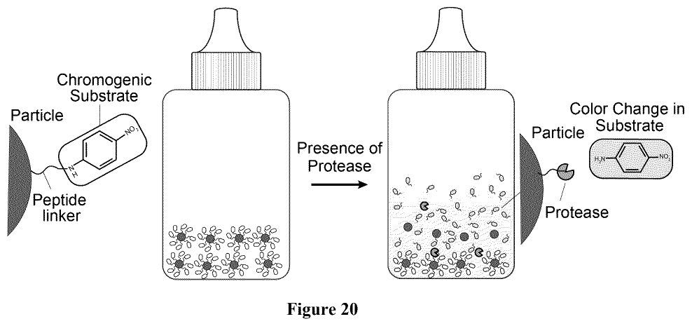

FIG. 20 is an example where the biocompatible fluid contains a reporter that, upon cleavage by the protease, will undergo a colorimetric change that will change the visual appearance of the solution. In this case, the color of the fluid will change from transparent to yellow when using the chromogenic substrate, p-nitroanilide. For example, certain molecules, including but not limited to p-nitroanilide, will undergo an electron resonance shift when cleaved from a peptide, which results in a color change in the solution upon cleavage.

In preferred embodiments, the reporter is non-toxic to humans at concentrations in which it may be introduced to the body through the biocompatible fluid. Examples of low-toxicity reporters include, but are not limited to, gold nanoparticles or nanoshells (e.g., BioReady carboxylic acid functional Gold Nanoshells by nanoComposix), iron oxide particles, fluorescein and its derivatives, and fluorescent or luminescent microbeads (e.g., fluorescent silica particles from CD Bioparticles or Fluoro-Max™ fluorescent carboxylate-modified particles supplied by ThermoFisher). In preferred embodiments, the biocompatible reporters contain at least one reactive group. Non-limiting examples include a carboxylic acid group, an activated ester group, an amine group, an alkene group, an isothiocyanate group, or a silane-reactive oxide surface (e.g., iron oxide or SiO2).

In many cases it is desirable for the reporter to be colorimetric or fluorescent in the visible spectrum, as this enables a change to be detected by the naked eye. In these cases, it is preferable for the fluid container to contain at least one optically transparent surface, such that the sensor can be observed by eye. In some embodiments, the sensor, which is visible through the one or more transparent surfaces of the fluid container, changes color in response to a protease-triggered cleavage of the peptides, while the color of the bulk solution does not change color in a way that can be detected by the naked eye.

In some embodiments in which the biocompatible fluid container is a fluid dispenser, the sensor may be contained in a chamber external to the main fluid container, through which a portion of the biocompatible fluid may be dispensed during use.

In some embodiments, the sensor is self-contained through a porous material or membrane with a pore size larger than the size of proteases, but smaller than the size of the reporters, such that protease may access the sensor, but the reporters do not diffuse into the bulk fluid in the container.

In a preferred embodiment using the architectures and chemistries disclosed herein, eye droppers, contact-solution containers, or contact-lens cases contain protease sensors for monitoring for bacterial contamination. When the sensors are placed in an eye-drop container, contact-solution container, or contact-lens case, they may be used to detect a potential contamination by Pseudomonas aeruginosa, Staphylococcus aureus, E. coli, or Listeria monocytogenes.

The eye drop container, contact-lens solution container, and contact-lens case sensors may be of the same peptide-based surface chemistries as described for the point-of-care diagnostic device for bacteria. However, the specific form factor of the sensor may preferably be different so as to be readily observed by the naked eye in the container or case.

The present invention includes devices and methods for improving the sampling of targeted biomarkers in tear fluid as a means to improve the signal-to-noise ratio of point-of-care ocular diagnostic devices, including lateral flow assays (LFAs). According to these embodiments, a wearable ocular insert is provided, which contains a covalently-bound capture agent that selectively targets the biomarker of interest in tear fluid. The ocular insert is combined with a release agent, which releases a component of the biomarker of interest (referred to here as the analyte). The released analyte is then detected by a point-of-care assay. The analyte may be a component of the captured biomarker or may be the entire captured biomarker, which has been released from the ocular insert.

In preferred embodiments, the wearable ocular insert is either a contact lens or punctal plug. However, the wearable ocular insert may be any object that can readily be placed and worn for extended periods of time (at least about 5 minutes) either on the surface of the eyeball or inside the conjunctival sac or lacrimal punctum of the human eye.

In preferred embodiments, the assay is a lateral flow assay comprised of capture and detection agents that detect at least one component of the released component of the biomarker captured by the ocular insert.

In other embodiments, the captured biomarkers may not be released, but the optical insert may be exposed to an indicator solution containing dyed molecules or particles conjugated to biological agents (for example, proteins, antibodies, aptamers, and the like) that bind specifically to the captured biomarker.

In preferred embodiments, the optical insert may be a contact lens, a punctal plug, or a device that can be placed on the eye for an extended period of time (at least 5 minutes) that will sample the infected ocular surface and tear film and collect, concentrate, and localize the targeted biomarker to the surface of the ocular insert.

When ocular insert is exposed to the indicator solution, the captured biomarker will become labeled with the dyed agent, resulting in an observable signal indicating the presence of the biomarker in the ocular surface or tear film.

As an example of this type of architecture, the surface of a silicone substrate may be modified to adhere to the positively charged surface of a virus common to ocular infection, Herpes simplex virus-1 (HSV1). The silicone substrate used in these experiments may be one that is biocompatible and has been formulated to be a major component in contact lenses. Upon exposure to solutions containing, for example, HSV1 and subsequent exposure to a solution containing nanoparticles conjugated with antibodies targeting HSV1 envelope proteins, a noticeable visual signal may be observed from the nanoparticles binding to captured HSV1 viral particles.

The use of a wearable ocular insert containing immobilized binding agents enables the targeted biomarkers of interest to be collected and concentrated for as long as the ocular insert is worn by the patient. This prolonged collection and concentration of biomarkers may increase the concentration of the bound biomarker on the surface of the ocular insert over the time the insert remains in the eye and greatly enhances the signal-to-noise ratio of the subsequent assay.

This invention is particularly well suited for improving the detection of ocular antigens such as viruses, bacteria, fungi, or amoeba. For example, viruses, such as the HSV-1 virus, contain many glycoproteins that are bound to the viral envelope. These glycoproteins (the analyte of interest) may be directly targeted by antibodies contained within a point-of-care diagnostic.

However, reliable detection of the virus in this way is challenging because many of the targeted glycoproteins may not be accessible to the antibodies of an LFA while the virus remains intact. Therefore, the invention describes two strategies to develop a sensor that may increase the sensitivity of the assay. In a first strategy, after the ocular insert captures the targeted pathogen (e.g., virus, bacteria), the insert may be added to a solution that breaks down the intact virus, releasing the targeted biomarkers on or within the pathogen, so it may more easily be captured and detected by the antibodies on an LFA. In a second strategy, after the ocular insert captures the targeted pathogen, the insert is placed into a solution containing a reporting agent that directly binds to the pathogen bound to the insert and causes a noticeable change in the appearance of the insert.

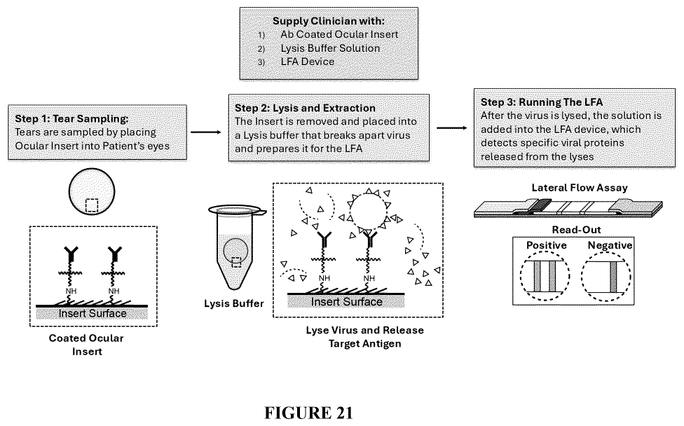

As a non-limiting example, FIG. 21 describes the release of the analyte by lysing the captured virus in a lysis buffer. This example illustrates the invention as the combination of an antibody-coated ocular insert, a lysis buffer, and an LFA device.

In some embodiments, the ocular insert contains a surface-immobilized blocking agent to prevent non-specific adsorption and fouling of the surface by other proteins and cells in the eye.

FIG. 21 is an example of a capture-and-release strategy for capturing HSV-1 virions and releasing HSV-1 analyte for detection on an LFA. One example of a suitable HSV-1 analyte is glycoprotein D. In a preferred embodiment, the architectures and chemistries disclosed herein may be used to provide a tool for point-of-care diagnosis of HSV-1 keratitis via an ocular-insert immunoassay.

An additional aspect of this invention is a diagnostic test comprised of a wearable ocular insert, which contains a covalently-bound capture agent organized on a portion of the ocular insert. In this embodiment, the ocular insert may be combined with a second solution that contains a colorimetric or fluorometric reporter bound to a detection antibody.

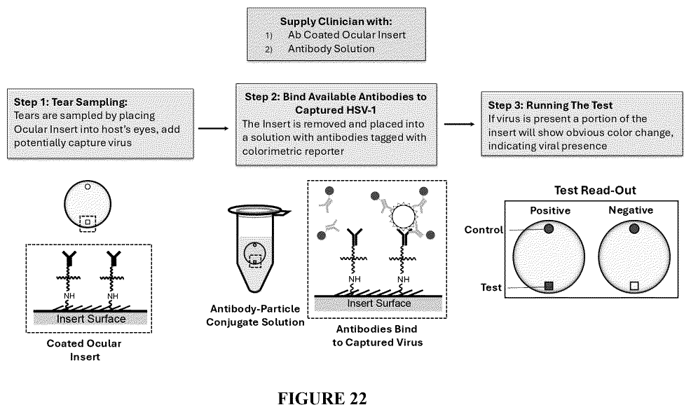

In preferred embodiments, the ocular insert may also contain a second region with a surface-immobilized antigen selected to react with the detection antibody (a control region). By having both a test region and a control region, the ocular insert may present readouts similar to a lateral flow device, as illustrated in FIG. 22.

As an example of this type of architecture, a silicone substrate may be formatted similar to that of a Schirmer strip. In this format, the silicone substrate may be able to sit in the lower fornix of the eye for an extended period of time (e.g., at least 30 seconds). A surface treatment may be applied to a region of the substrate to greatly increase its affinity towards the virus. In this example, an oxygen plasma may be used to render the silicone substrate hydrophilic with a negative surface charge. The plasma treated silicone may bind to the virus with a higher affinity as compared to the as-prepared silicone surface.

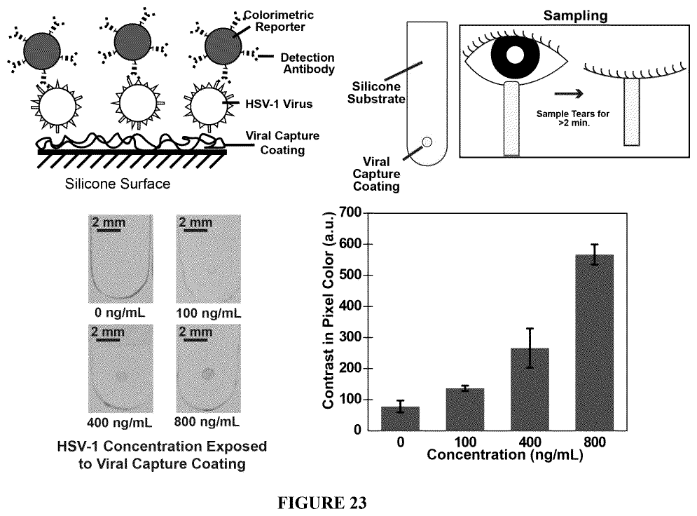

The silicone substrate may be exposed to dilutions of the herpes simplex virus 1 (HSV-1). The substrate may then be exposed to a solution containing gold nanoshell particles conjugated with anti-HSV-1 antibodies, such that these particles may bind to the HSV-1 captured by the silicone substrate. After a rinsing procedure, which removes the gold particles not adhered to the captured HSV-1, the substrates may be imaged and analyzed for a difference in color of the capture zone relative to the surrounding substrate. In this example, HSV-1 solutions may be detected down to 100 ng/mL, as shown in the experimental results reported in FIG. 23. This approach may be applied to detect other types of viruses, bacteria, fungi, proteins, or antibodies that may be secreted into the tear film.

In another embodiment, the areal density of the capture agents is significantly greater than the density of a smooth monolayer of the capture agent. The effective thickness of the area in which the capture agent is immobilized is critical in order to increase the areal density of reporters in the test region, thereby achieving a low limit of detection and easy readout by the naked eye. A layer of capture agents with an effective thickness greater than a monolayer may be achieved by using a water-permeable ocular insert wherein the pore size of the permeable material is greater than about five times the diameter of the targeted biomarker, wherein the capture agent is bound to substantially all inner surfaces of the permeable material.

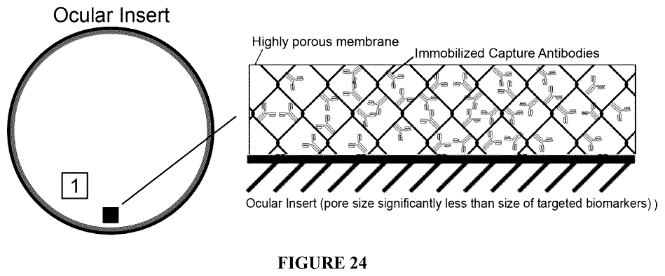

Alternatively, the ocular insert may be primarily comprised of a material with a pore size less than the size of targeted biomarkers, but may contain a layer of water-permeable material with a pore size greater than the size of the targeted biomarkers, laminated to the test region and, optionally, also laminated to the control region, as shown in FIG. 24. In this embodiment, the thickness of the laminated layer is at least about 10 times greater than the size of the targeted biomarker.

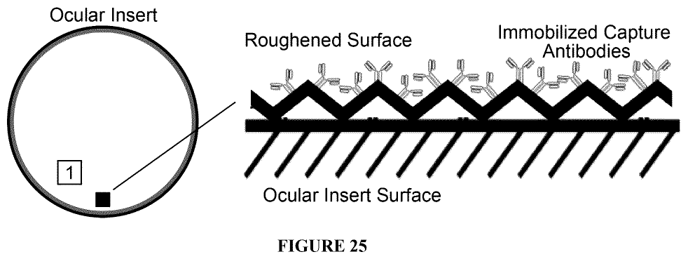

In another embodiment illustrated in FIG. 25, the test region (and, optionally, the control region as well) of the ocular insert contains a roughened surface where the root-mean-square roughness is about 10 times greater than the size of the targeted biomarker. For example, for capturing the HSV-1 virus (with an approximate diameter of 100-200 nm), the root-mean-square roughness is at least about 1-2 microns. This surface roughness may be increased by physical or chemical etching of the surface of the ocular insert, deposition of a monolayer or multilayer of micro or nanoparticles, or lamination of a porous film or foam, as well as by photolithography, nano-imprinting, micro-imprinting, or 3D printing a roughened surface.

The use of a porous or roughened surface to immobilize the capture agents increases the density of the capture agents and, consequently, enables a larger concentration of biomarkers to be captured before the ocular insert is fully saturated. This is critical for enabling a high signal-to-noise ratio when the colorimetric or fluorometric reporters are added in the second step of the diagnostic test.

The reporters of the detection solution are either colorimetric agents such as pigments that absorb or reflect a substantial portion of the visible spectrum (including, but not limited to, gold nanoparticles, iron oxide particles, carbon black, or activated charcoal), dyes, or fluorometric agents, such as fluorescent dyes, quantum dots, or luminescent particles (including luminescent rare-earth elements).

In embodiments utilizing colorimetric reporters, it is preferred that the test region contains a material that provides visible contrast. Visible contrast may be imparted via light scattering, which may be provided by utilizing porous or highly roughened surfaces, such as those shown in

FIGS. 3 and 4, or by utilizing pigments, dyes, or surfaces that absorb a portion of the visible spectrum that is significantly different than the portion of the visible spectrum absorbed by the colorimetric reporter. For example, if the reporter is a black pigment, the test region may be permanently dyed any color that can readily be distinguished from black (e.g., blue, green, or red).

This aspect of the invention may be especially advantageous for ocular inserts that are typically made out of transparent materials, such as silicone punctal plugs or silicone or poly (HEMA) contact lenses. Without a supporting material that provides visible contrast, the color change may be too faint to be readily and reliably detected for a proper point-of-care test.

Alternatively, easy naked-eye readout may be achieved by utilizing a fluorescent or luminescent reporter with emission in the visible spectrum. To maximize contrast, it is preferable that the surface of the test and control portions of the ocular insert be selected to have minimal auto-fluorescence.

In preferred embodiments, the ocular insert contains a surface-immobilized blocking agent to prevent non-specific adsorption of the antibody-bound reporters. Examples of blocking agents include, but are not limited to, proteins, such as bovine serum albumin or casein, or polyethylene glycol.

Another aspect of this invention is a method in which a mucolytic agent is first applied to the ocular surface prior to sampling and/or performing a diagnostic assay of the tear film or ocular surface.

During an ocular infection, a significant amount of the infection agent may be in the mucin layer and the glycocalyx just above the epithelial layer of the ocular surface. Traditional eye swabs or other type of ocular sampling or detection tools may have difficulty accessing infection agents or other biomarkers related to the infection agent contained within these layers. Releasing the infection agent or their related biomarkers from these layers prior to or during sample collection or during direct detection may enhance the signal of the assay of the infection agent or biomarker.

One approach to release a targeted biomarker from these layers is through a mucolytic agent, which changes the mucin layer and/or glycocalyx to release biomarkers or infection agents within these layers. The infection agent or biomarker will then be more accessible to an ocular swab, Schirmer strip, or any other type of ocular insert used for sampling the tear film or ocular surface. Examples of suitable mucolytic agents for this application include, but are not limited to, N-acetylcysteine (NAC), carbocysteine, sobrerol, Letosteine, iodinated glycerol, N-isobutyrylcysteine (NIC), and Erdosteine.

In preferred embodiments, the mucolytic agent is applied to the eyes via an eye dropper, a period of time of approximately 10 seconds to 15 minutes is allowed to pass prior to sampling the ocular surface, the ocular surface or tear film is then sampled, and an assay for the targeted infection agent is performed.

Examples of ocular sampling tools that may be used for this method include, but are not limited to, ocular swabs, Schirmer's strips, ocular scraping tools, or any of the ocular inserts described herein. Examples of assays performed using this method include, but are not limited to, immunoassays (e.g., ELISA), lateral flow assays, PCR, RT-PCR, mass spectrometry, gram staining and bacterial, fungal, and viral culture.

Although the descriptions of this invention are particularly suited towards ocular inserts, the architectures and surface chemistries described may be used on any material that can be readily applied to any infection on the body (for example, cold sores).

FIG. 22 is an example of an ocular insert immunoassay containing a test and control area. In preferred embodiments, one of at least the shape or size of the test and control areas are substantially different from one another, enabling the user to readily distinguish the two by eye.

FIG. 24 is an example of an ocular insert wherein the active region (1) contains a porous membrane containing covalently-bound capture antibodies laminated to the surface of an ocular insert. The pore size of the membrane is much larger than both the capture antibodies as well as the targeted biomarker of interest.

FIG. 25 is an example of an ocular insert wherein the active region (1) contains capture antibodies immobilized on a roughened surface on a portion of an ocular insert. This figure is not drawn to scale, as in actuality the roughness is much larger than the size of the capture antibodies.

In further embodiments, a contact lens may contain one or more test regions to detect one or more different biomarkers. In cases where more than one test region is embedded in the contact lens, the regions may preferably be separated by a distance large enough to be readily distinguishable by the naked eye. The shape of each test region may be, for example, rectangular, circular, an annual ring around the contact lens or a portion thereof, or any shape that is large enough to be observed by the naked eye (at least about 50 microns in observable area).

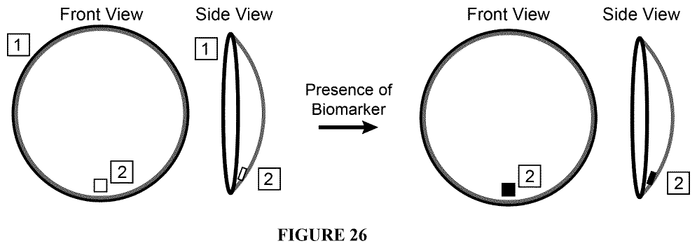

Several examples of different layouts of the ocular insert are shown in FIGS. 26-30. In some embodiments, the one or more sensors are embedded on the convex surface of the contact lens. In other cases, it is preferable for the sensor to be on the concave side of the lens that directly contacts the eye. Such an architecture is particularly preferable for cases where the contact lens is to be used as a point-of-care device for diagnosing corneal ulcers. Embedding the test region on the concave surface that directly contacts the epithelium of the eyeball enables the test region to be placed in direct contact, or in the immediate vicinity, of the inflamed surface of the eye for cases of keratitis. This architecture greatly simplifies the procedure required to sample corneal ulcers, which traditionally are sampled for lab culture by first scraping the top surface of the cornea or conjunctiva where the ulcer is observed. Placing the one or more sensors on the concave side of the contact lens circumvents the need for a corneal scraping as it comes in direct contact with the eye. For purposes of simplifying the manufacturing, it may be preferable for the test region to coat the entire (both concave and convex) surface of the contact lens.

FIG. 26 is an example of a colorimetric or fluorometric test region (2) for testing one biomarker attached to the surface of a contact lens (1). Although not shown in this figure, in preferred embodiments, the test region is paired with a control region such as is elsewhere described herein. The test region (2) may bind the targeted biomarker and then, through the result of an assay, the captured biomarker will become detectable (e.g., change its observed color or become fluorescent).

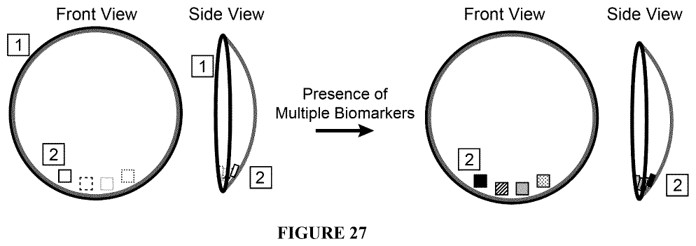

FIG. 27 is an example of multiple colorimetric indicators (2) attached to the surface of a single contact lens (1), each of which tests for a different biomarker in order to target a different ocular pathogen. Although not shown in the figure, in preferred embodiments, each test region is paired with a control region containing an immobilized antigen selected to bind with the detection antibody.

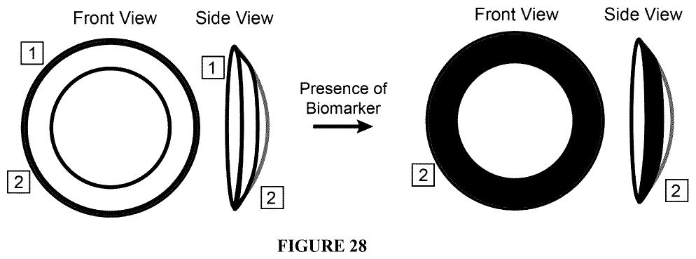

FIG. 28 is an example of a colorimetric indicator test region on the surface of a contact lens (1) in an annular shape (2) around the lens itself.

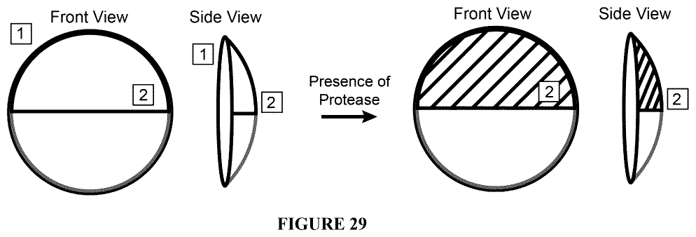

FIG. 29 is an example of a colorimetric indicator (2) occupying a larger section of the contact lens (1), which may be anywhere from about 10%-100% of the total area of the lens.

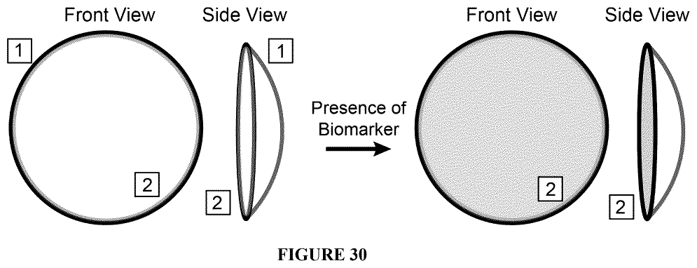

FIG. 30 is an example of a contact lens (1) with an unmodified lens surface (front, convex side) and with a concave side (2) containing the embedded test and control regions, with a covalently-bound capture agent.

In some embodiments, the contact lens may be designed to contain a protrusion extending outward from the convex surface of the contact lens to enable easy insertion in the eye without directly touching the surface of the contact lens which contains the protease-specific peptides and reporters. To further aid with the insertion and removal of the lens without damaging or contaminating the sensor region, the ocular insert may contain permanently embedded magnetic particles, such as iron oxide. Such a contact lens may be inserted and removed using any object with a magnetic tip.

In some embodiments, the ocular insert may be in the form of a punctal plug, or any object which can readily be placed and worn on the surface of the eyeball or inside the conjunctival sac or lacrimal punctum of the human eye. For example, the insert can be a string of cellulosic material, a disc or crescent shaped material that is no more than about 5 mm in at least two orthogonal directions.

In some embodiments, the ocular insert may contain magnetic particles, enabling the insert to be inserted and removed from the eye using a magnet.

In some embodiments, the ocular insert may be a Schirmer's strip, as described above. In this modified version of a Schirmer's strip, a section of the Schirmer's strip may be chemically modified to contain a test region comprised of the one or more protease-responsive reporters. Preferably, this test region is positioned within a distance of 0 mm to about 20 mm away from the end of the strip to be inserted into the conjunctival sac. A Schirmer's strip is advantageous because it is widely utilized by, and therefore familiar to, optometrists and ophthalmologists. However, the peptide-bound ocular insert can be any object that is readily inserted or removed from the conjunctival sac. For example, instead of a Schirmer's strip, the insert can be comprised of a string of cellulosic material that is modified with the peptide-bound reporter. Alternatively, it may be a disc or crescent shaped hydrogel that is crosslinked solely by the protease-responsive peptides.

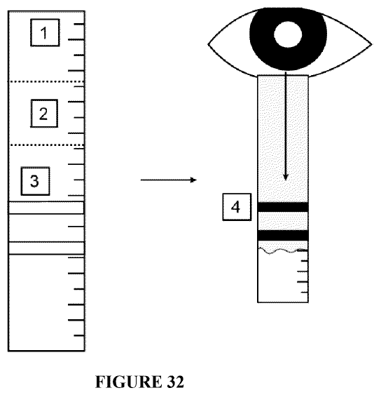

FIG. 32 is an example of an LFA Schirmer's strip. Similar to a traditional LFA, the LFA Schirmer's strip is comprised of: a sample pad, a conjugate pad, and a test pad containing a test line and control line.

In preferred embodiments, the conjugate and test pad are encased in a water impermeable transparent film including, but not limited to, PET, an acrylic copolymer, LDPE, or silicone. Each component of the LFA is thin (less than about 0.2 mm) and lightweight, enabling it to be used as a Schirmer's strip. The sample pad is placed in the conjunctival sac of the patient and tears flow down past the conjugate pad onto the sample pad. In a positive test, both the test and control lines change color.

What is claimed is:

1. An ocular insert containing one or more reporters:
wherein each of the reporters is bound either directly or indirectly to the ocular insert via a peptide sequence that is selectively cleaved by one or more targeted proteases,
and wherein each protease-cleavable peptide sequence is hydrolytically stable in tear fluid for at least about 30 minutes in the absence of the targeted protease.

2. The ocular insert of claim 1, wherein the one or more reporters are compounds or materials that are non-toxic to humans at the concentrations in which they may be released in the eye when the peptide is cleaved.

3. The ocular insert of claim 1, wherein one end of the protease-cleavable peptide is covalently bound to the reporter and another end of the peptide is adhered to a surface of the ocular insert.

4. The ocular insert of claim 1, wherein the one or more targeted proteases are secreted by one or more specific species of bacteria, amoeba, or fungus.

5. The ocular insert of claim 1, wherein at least one of the reporters is a protease specific-reporter that is an orthogonal photoacoustic probe, comprised of one photoacoustic moiety bound to the surface of the ocular insert via a chemical bond that is stable in both the presence and absence of the one or more targeted proteases, and a second photoacoustic moiety covalently bound either directly or indirectly to the surface of the ocular insert via the protease-cleavable peptide, and wherein the photoacoustic signals of the two photoacoustic moieties are orthogonal to one another.

6. The ocular insert of claim 1, wherein at least one of the reporters is a visible-light absorbing pigment, dye, or particle.

7. The ocular insert of claim 1, wherein at least one of the reporters includes a fluorescent dye, pigment, particle, or bead.

8. The ocular insert of claim 1, wherein the ocular insert further comprises a first surface with a first color detectable by the naked eye and a second surface containing the one or more protease cleavable peptide-bound colored moieties, wherein the one or more protease cleavable peptide-bound colored moieties have a second color that is different from the first color, and wherein the second surface visibly blocks the first color of the first surface until the peptide is cleaved by a protease, thereby revealing the second color.

9. The ocular insert of claim 1, wherein the ocular insert further comprises a first region containing a permanently embedded or bonded reporter, and a second region, on top of the first region, containing a second protease-responsive, peptide-bound reporter.

10. The ocular insert of claim 9, wherein the first region is oriented in a shape that is different and distinguishable by the naked eye from the second region.

11. The ocular insert of claim 1, wherein the ocular insert is a contact lens or punctal plug.

12. The ocular insert of claim 1, wherein the ocular insert is a cylinder, disc, ellipsoid, prismatic, or crescent shaped object that is between about 2 mm to 20 mm long in at least one direction and no more than about 2 mm thick.

13. The ocular insert of claim 1, wherein the ocular insert is a water-permeable strip or string of material at least about 5 mm long and no more than about 2 mm thick.

14. The water-permeable strip of claim 13, wherein the water-permeable strip is comprised primarily of a cellulosic material.

15. The ocular insert of claim 1, wherein at least one of the targeted proteases is secreted by at least one of Staphylococcus aureus, Streptococcus pneumoniae, bacillus, Pseudomonas aeruginosa, enterobacteriaceae, Neisseria gonorrhoeae, morraxella spp., or Haemophilus influenzae.

16. The ocular insert of claim 1, wherein at least one of the targeted proteases is secreted by at least one of aspergillus, candida spp., cryptococcus species, fusarium, penicillium, pseudallescheria, dimorphic fungi as Histoplasma capsulatum, Blastomyces dermatitidis, sporothrix spp., and coccidioides spp.

17. The ocular insert of claim 1, wherein at least one of the targeted proteases is secreted by acanthamoeba.

18. The ocular insert of claim 1, wherein at least one of the targeted proteases is selected from Elastase A (LasA), Elastase B (LasB), Alkaline Protease (AP), or Protease IV (PIV).

19. A diagnostic test comprising:
an ocular insert with one or more surface immobilized binding agents for one or more target ocular biomarkers;
a mechanism for releasing the targeted analytes from the ocular insert; and
a lateral flow assay that detects the one or more released analytes.

20. The diagnostic test of claim 19, wherein, at least one of the surface immobilized binding agents specifically binds to one or more surface proteins on the targeted biomarker.
